(19) United States
(12) Patent Application Publication
(10) Pub. No.: US 2022/0249276 A1
(43) Pub. Date: Aug. 11, 2022

(54) Mare Intrauterine Device

(71) Applicant: WiSys Technology Foundation, Inc., Madison, WI (US)

(72) Inventors: Casie Bass, JR., Spring Valley, WI (US); John Obielodan, Platteville, WI (US)

(21) Appl. No.: 17/669,894

(22) Filed: Feb. 11, 2022

Related U.S. Application Data
(60) Provisional application No. 63/148,322, filed on Feb. 11, 2021.

(51) Int. Cl. A61F 6/18; A61F 6/14

(52) U.S. Cl. CPC A61F 6/18; A61F 6/142

(57) ABSTRACT

An intrauterine device (IUD) is insertable into a uterus of a mare to suppress estrus and estrus-related behaviors in mares. The IUD may take the form of a weighted ball or sphere that is biocompatible and includes a string attached to the weighted ball for ease of removal by pulling down on the string.

### CROSS REFERENCE TO RELATED APPLICATION

This application claims the benefit of U.S. Provisional Patent Application No. 63/148,322, filed Feb. 11, 2021, which is incorporated by reference herein in its entirety.

### STATEMENT REGARDING FEDERALLY SPONSORED RESEARCH OR DEVELOPMENT

--

### BACKGROUND OF THE INVENTION

The present invention relates generally to regulating the estrous cycle in mares and, in particular, to a contraceptive device that is inserted into the uterus of the mare to prevent pregnancy.

Female horses or other equine animals, such as mules and zebras, that are older than four years old are called “mares.” The estrous cycle in most mares normalizes in early spring to autumn, which is the normal breeding season for horses. During the cycles, the mare will have an estrous cycle of approximately 19 to 22 days composed of the estrus phase (i.e., in heat) and diestrous phase (i.e., out of heat). Ovulation, the release of the egg from the ovary, occurs any time during the estrus phase but normally occurs 24 to 48 hours before the end of the estrus period. The estrus phase is characterized by a spike in estrogen and estrus-related behaviors that lasts for about a week. The diestrous phase is characterized by a spike in progesterone phase that lasts for about two weeks.

Estrus-related behaviors during “heat” may include moody or distracted behavior. Owners of mares may desire to suppress undesirable estrus-related behaviors in horses.

Oral administration of progestin altrenogest, e.g., Regu-Mate, has been one method to suppress estrus but progestin altrenogest may be absorbed into the human skin, potentially disrupting menstrual cycles or causing miscarriages in humans, or contaminating the environment. Another method has been injectable progesterone products that must be injected regularly into the mare, leading to soreness at the injection site.

Intrauterine devices have also been implemented by, for example, placing a glass marble into the mare's uterus causing the uterus to mimic pregnancy, i.e., to mimic an embryo and to induce chronic uterine inflammation and low-grade prostaglandin (PG) release. This causes the mare to maintain and reform the corpus luteum (CL) thereby maintaining progesterone concentrations and pregnancy. However, some mares suffer major long-term ramifications if the marbles are left in too long (e.g., longer than 18 months) affecting their ability to breed or will prematurely eject the marbles before they can prevent estrus. The marbles may also cause inflammation and infection, may break, or may undesirably adhere to the endometrium.

Current methods of suppressing undesirable estrus-related behaviors have had limited success with unwanted drawbacks.

### SUMMARY OF THE INVENTION

The present invention provides an intrauterine device (IUD) insertable into a uterus of a mare to suppress estrus and estrus-related behaviors in mares. The IUD may take the form of a weighted ball or sphere that is biocompatible and includes a string attached to the weighted ball for ease of removal by pulling down on the string.

It is thus one feature of one embodiment of the present invention to provide an IUD that is shatterproof, is well retained within the uterus, and removed after 3 to 18 months using an attached string.

In one embodiment of the present invention, the device may be a fluid-filled medical grade plastic ball.

In an alternative embodiment of the present invention, the device may be a wooden ball coated in a medical grade plastic.

In an alternative embodiment of the present invention, the device may be a hollow medical grade elastic ball that is fillable, and refillable, with a fluid of desired weight.

One embodiment of the present invention provides an intrauterine device for insertion into a uterus of a mare to regulate estrous cycle including a hollow ellipsoid having a cavity and sized to be insertable into the uterus of the mare; a string attached to the ellipsoid; a filler filled within the cavity of the hollow ellipsoid wherein the filler is denser than a material of the hollow ellipsoid and wherein the ellipsoid has a greatest outer diameter between 25 and 40 mm and a weight of at least 4 g.

It is thus a feature of at least one embodiment of the present invention to provide an intrauterine device that is shaped to be easily inserted but retained within a uterus of a mare but can be easily removed by pulling on an attached string when desired.

The cavity may have a cavity volume of at least 0.45 ml.

It is thus a feature of at least one embodiment of the present invention to allow for a weighted filler material to be filled in the cavity of the sphere to provide a desirable weight to mimic an embryo and induce chronic uterine inflammation and low-grade prostaglandin (PG) release.

The filler may be a fluid having a volume of at least 0.2 ml. The fluid may have a density of at least 1 g/ml.

It is thus a feature of at least one embodiment of the present invention to easily adjust the weight of the sphere by changing the amount of the filler and type of filler with varied densities.

The filler may be selected from the group consisting of medical grade silicone, wood, saline solution, and brine.

It is thus a feature of at least one embodiment of the present invention to use high density materials that are biocompatible and increasing the weight of the sphere without increasing the volume of the sphere.

The string may have a length of at least 10 cm. The string may have a length of at least 20 cm.

It is thus a feature of at least one embodiment of the present invention to allow the string to extend outwardly from the mare's uterus or vagina and is easily retrievable to be pulled out without having to pull the sphere out from inside the uterus or vagina.

The string may be selected from the group consisting of rayon and rayon-cotton blend.

It is thus a feature of at least one embodiment of the present invention to prevent breakage of the string when it is being forcibly pulled to remove the sphere from the uterus.

The hollow ellipsoid may be at least two joinable components.

It is thus a feature of at least one embodiment of the present invention to allow the device to be filled with a dense fluid before insertion into the mare to mimic embryo weight.

The at least two joinable components may be two spherical caps. The two spherical caps may be approximately equal halves of a sphere.

It is thus a feature of at least one embodiment of the present invention to vary or maximize the amount of fluid that may be filled within the spherical device thus varying or increasing the weight of the sphere.

The at least two joinable components may be joined through screw threads. The intrauterine device may further comprise a rubber gasket positioned between the two joinable components.

It is thus a feature of at least one embodiment of the present invention to prevent leakage of the fluid from the device when inside the uterus by providing a watertight seal between the joinable parts.

The two joinable components may be joinable through a friction fit. The two joinable components may be joinable through a snap fit.

It is thus a feature of at least one embodiment of the present invention to allow the components to be easily joined by hand and without additional tools.

The hollow ellipsoid may be a unitary body.

It is thus a feature of at least one embodiment of the present invention to minimize leaks from the sphere.

The hollow ellipsoid may have at least one hole. The string may be attachable to the ellipsoid by extending through the at least one hole and tied to secure the string.

It is thus a feature of at least one embodiment of the present invention to securely join the string to the device while still providing a smooth exterior to the device that minimizes tissue irritation.

The hole may be selectively closeable by at least one plug.

It is thus a feature of at least one embodiment of the present invention to permit the filler to be filled within the cavity through small holes in the device which prevent leakage.

The intrauterine device may further comprise a loop attached to an outer surface of the ellipsoid. The string may be securely attached to the loop.

It is thus a feature of at least one embodiment of the present invention to securely join the string to the sphere so that it is not accidentally separated from the sphere when the string is pulled.

A wall of the hollow ellipsoid may be between 1.5 mm and 8 mm. The ellipsoid may be selected from the group consisting of PVC, Polyethylene, PEEK, Polycarbonate, Ultem PEI, Polysulfone, Polypropylene, Polyurethane, any suitable polymer material, any suitable biocompatible material, and any suitable non-biocompatible material that is properly coated with biocompatible material.

It is thus a feature of at least one embodiment of the present invention to provide a biocompatible material that will not break or separate inside the uterus after an extended period of time.

One embodiment of the present invention provides a method of inserting an intrauterine device into a uterus of a mare comprising: providing a hollow ellipsoid insertable into the uterus of the mare. A string is fixedly attached to the ellipsoid and facilitates removal of the ellipsoid from the uterus of the mare and a fluid is filled within a cavity of the hollow ellipsoid and has a weight wherein the ellipsoid has a greatest outer diameter between 25 and 40 mm. The method comprises further filling the fluid within the cavity of the hollow ellipsoid; inserting the hollow ellipsoid into the uterus of the mare, and pulling on the string of the hollow ellipsoid to remove the hollow ellipsoid from the uterus of the mare.

These particular objects and advantages may apply to only some embodiments falling within the claims and thus do not define the scope of the invention.

### BRIEF DESCRIPTION OF THE DRAWINGS

FIG. 1 is an exploded perspective view of the intrauterine device (IUD) of the present invention insertable into a uterus of a mare for suppression of estrus;

FIG. 2 is a phantom side elevation view of the IUD of FIG. 1 showing the first capsule part attached to a second capsule part via a screw attachment and gasket;

FIG. 3 is a cross sectional view of the IUD of FIG. 1 showing the first capsule part and second capsule part attached and filled with a fluid;

FIG. 4 is an exploded perspective view of a second embodiment of the intrauterine device (IUD) of the present invention insertable into a uterus of a mare for suppression of estrus;

FIG. 5 is a phantom side elevation view of the IUD of FIG. 4 showing the first capsule part attached to a second capsule part via a screw attachment and gasket;

FIG. 6 is a cross sectional view of the IUD of FIG. 4 showing the first capsule part and second capsule part attached via a screw attachment and filled with a fluid;

FIG. 7 is an exploded perspective view of a third embodiment of the intrauterine device (IUD) of the present invention insertable into a uterus of a mare for suppression of estrus;

FIG. 8 is a cross sectional side elevation view of the IUD of FIG. 7 showing the first capsule part and second capsule part attached via a friction fit and filled with a fluid;

FIG. 9 is an exploded perspective view of a fourth embodiment of the intrauterine device (IUD) of the present invention insertable into a uterus of a mare for suppression of estrus showing push plugs and string removed from openings of the unitary capsule to allow for fluid to be filled into the hollow capsule;

FIG. 10 is a cross sectional side elevation view of the IUD of FIG. 9 showing the push plugs and string attached to the openings of the unitary capsule and the hollow capsule filled with a fluid; and

FIG. 11 is a cutaway perspective view of a fifth embodiment of the intrauterine device (IUD) of the present invention insertable into a uterus of a mare for suppression of estrus having an outer layer and an inner layer.

### DETAILED DESCRIPTION OF THE PREFERRED EMBODIMENT

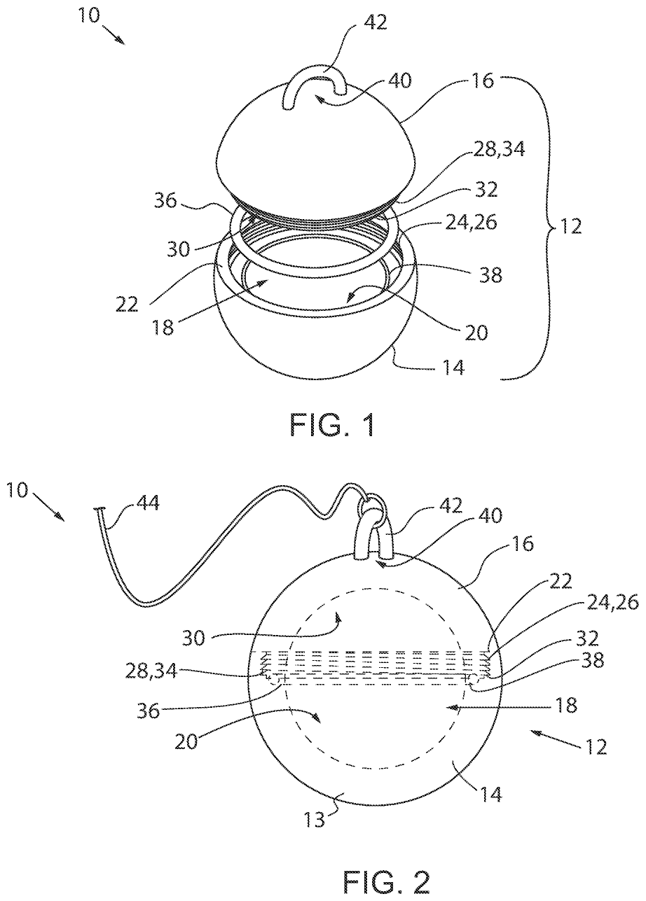

Referring now to FIG. 1 through FIG. 3, an intrauterine device (IUD) 10 of the present invention may provide a spherical body 12 that may be insertable into a uterus of a mare. The IUD 10 may be inserted using an applicator (not shown) to facilitate the insertion of the IUD 10 into the uterus of the mare. It is understood that the spherical body 12 may be an ellipsoid that may alternatively take an oblong or oval shape which may generally assist with insertion of the IUD 10 into the uterus of the mare.

In one embodiment of the present invention, the spherical body 12 may by defined by a first capsule part 14 and a second capsule part 16 that form the spherical body 12 when coupled together. The spherical body 12 is hollow such that the wall 13 of the first capsule part 14 and second capsule part 16 when joined define therebetween an interior spherical cavity 18. The first capsule part 14 is insertable into the uterus first, and is attachable to the second capsule part 16, which is insertable into the uterus second and positioned proximate the uterus opening or cervix of the mare once inserted.

The first capsule part 14 may provide a lower spherical cap that is hollowed out to provide an interior bowl 20. An interior surface 24 of an upper edge 22 of the first capsule part 14 may include screw threads 26 (e.g., female threads) that receive corresponding screw threads 28 (e.g., male threads) of the second capsule part 16. It is understood that the female threads and male threads may be interchanged with respect to the first and second capsule parts.

In one embodiment, the wall 13 of the first capsule part 14 may have an outer radius of between 15-20 mm and approximately 15 mm and approximately 17.50 mm, and an inner radius of between 10-15 mm and approximately 12.50 mm, such that the wall 13 is between 1.5-8 mm and between 3-8 mm and approximately 5 mm.

The second capsule part 16 may provide an upper spherical cap that is hollowed out to provide an interior bowl 30. A lower edge 32 of an exterior surface 34 of the second capsule part 16 may include screw threads 28 (e.g., male threads) that are received by the corresponding screw threads 26 (e.g., female threads) of the first capsule part 14. It is understood that the female threads and male threads may be reversed with respect to the first and second capsule parts.

In one embodiment, the wall 13 of the second capsule part 16 may have an outer radius of between 15-20 mm and approximately 15 mm and approximately 17.50 mm, and an inner radius of between 10-15 mm and approximately 12.50 mm, such that the thickness of the wall 13 is between 1.5-8 mm and between 3-8 mm and approximately 5 mm.

The first capsule part 14 and second capsule part 16 may be substantially halves of the sphere and similar in size. The first capsule part 14 and second capsule part 16 may be made out of a biocompatible and medical grade plastic material such as PVC, Polyethylene, PEEK, Polycarbonate, Ultem PEI, Polysulfone, Polypropylene and Polyurethane and the like. It is understood that the material may be any suitable polymer material, any suitable biocompatible material, or any suitable non-biocompatible material that is properly coated with biocompatible material.

In order to join the first capsule part 14 and the second capsule part 16, the second capsule part 16 may be finger tightened without the use of tools to the first capsule part 14 by inter-engaging threads 26, 28 such that the lower edge 32 of the second capsule part 16 is brought into the interior bowl 20 of the first capsule part 14 and abuts an inwardly extending rim 38 of the first capsule part 14 positioned just below the screw threads 26.

The inwardly extending rim 38 may support thereon a gasket material 36 such as an O-ring sealing the interface between the inwardly extending rim 38 of the first capsule part 14 and the lower edge 32 of the second capsule part 16. In one embodiment, the O-ring may have an outer diameter that generally corresponds to an inner diameter of the first capsule part 14 and may be between 25-20 mm and approximately 28 mm and approximately 28.3 mm. The O-ring may be made from PTFE, Nitrile (Buna), Neoprene, EPDM Rubber and Fluorocarbon (Viton) and the like.

It is understood that any type of connection method may be used to attach and seal the first capsule part 14 and the second capsule part 16 together. In an alternative embodiment of the present invention, the screw threads may be replaced with a press fit connection, friction fit connection, or snap fit connection between the first capsule part 14 and the second capsule part 16.

A string end 40 of the second capsule part 16 may include an outwardly projecting loop or ring 42, or alternatively a pad eye with a metal plate supporting the loop or ring 42, attached to the string end 40, that receives a hanging cord or string 44. The string 44 is tied, knotted, or otherwise securely attached to the ring 42. The string 44 may be made from a rayon or a rayon-cotton blend material and the like. The string 44 may extend a length approximating a distance between the vagina and uterus of the mare and may be at least 10 cm and at least 20 cm in length. The string 44 may be pulled outward from the vagina to easily remove the spherical body 12 from the uterus of the mare without having to reach into the vagina. The string 44 may be short enough, however, so that it is not accidentally pulled and to prevent accidental removal of the spherical body 12.

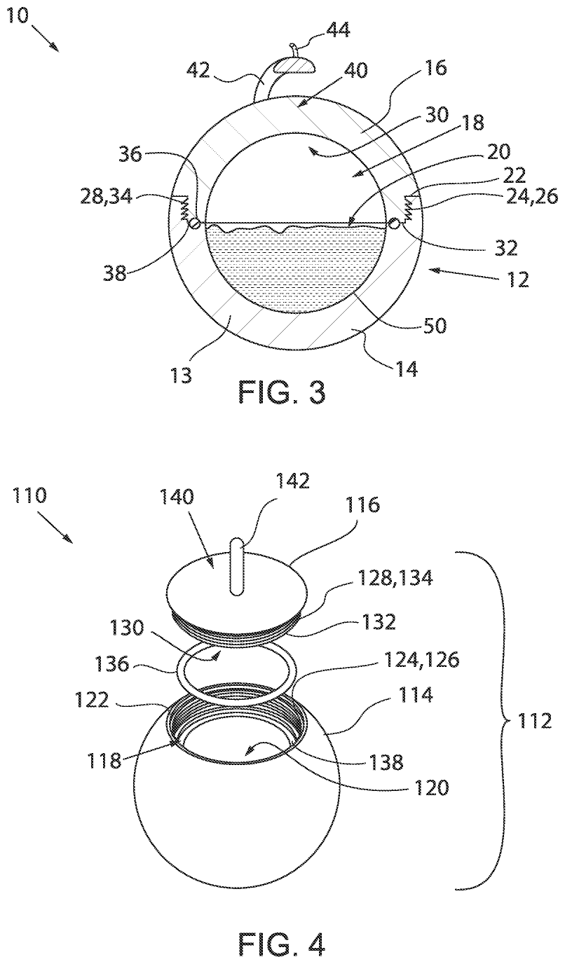

Referring specifically to FIG. 3, in use, the interior bowl 20 of the first capsule part 14 may be filled with a fluid 50 prior to the attachment of the second capsule part 16 to form the spherical body 12. Once the first capsule part 14 and second capsule part 16 are sealably attached, the spherical body 12 may be rotated or flipped such that the fluid 50 flows to fill the interior spherical cavity 18 of the second capsule part 16. The spherical cavity 18 may have a volume that is between 0.45 ml and 1.3 ml and is at least 0.45 ml and is at least 0.55 ml and is at least 0.65 ml and is at least 0.75 ml and is at least 0.85 ml and is at least 1 ml. A fluid volume of between 0.2 and 0.65 ml and at least 0.2 ml and at least 0.3 ml and at least 0.4 ml and at least 0.5 ml and at least 0.6 ml may be filled into the spherical cavity 18 therefore at least one-half and at least two-thirds and at least three-fourths of the cavity volume of the spherical cavity 18 may be filled with fluid 50.

The first capsule part 14 may be inserted into the uterus first, and the second capsule part 16 is positioned proximate the uterus opening or cervix. The string 44 may extend from the spherical body 12 and extend outwardly from the uterus and vagina for ease of removal of the spherical body 12 from the uterus.

The type of fluid 50, characteristics of the fluid 50, or density of the fluid 50 may provide a desirable weight of the spherical body 12. In one embodiment of the present invention, the fluid 50 is a medical grade water, silicone, saline solution, brine, and the like. The density of the fluid 50 may be between 1 g/ml and 1.045 g/ml and at least 1 g/ml. Generally, the density of the fluid 50 may be greater than the density of the material of the spherical body 12. In one embodiment, the density of the fluid 50 may be at least twice as dense as the material of the spherical body 12.

It is understood that the fluid 50 of the spherical body 12 provides the desired weight to the spherical body 12. In one embodiment of the present invention, the spherical body 12 has a weight between 4 g and 18 g and at least 4 g and at least 5 g and at least 6 g and at least 7 g and at least 8 g and at least 9 g and at least 10 g and at least 11 g and at least 12 g and at least 13 g and at least 14 g and at least 15 g and at least 16 g and at least 17 g.

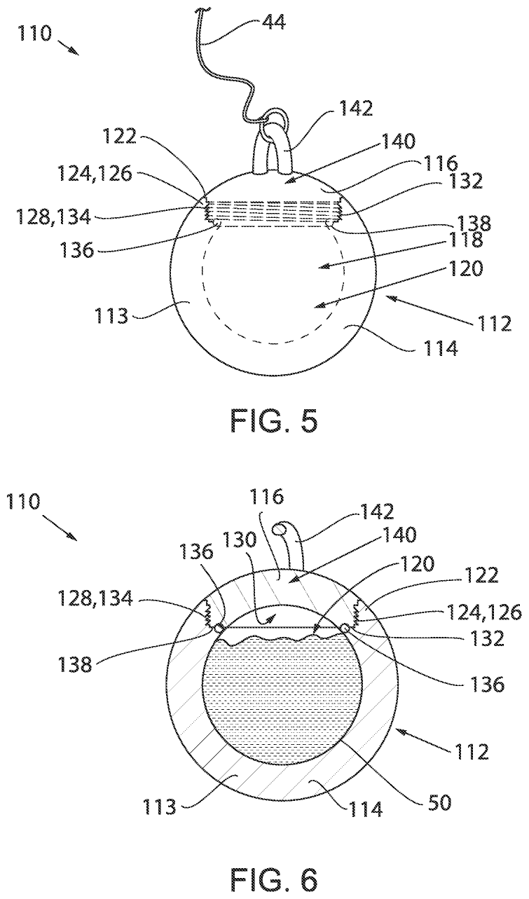

Referring to FIG. 4 through FIG. 6, an IUD 110 of a second embodiment of the present invention is similar to the embodiment shown in FIGS. 1 through 3, where similar parts are identified by like reference numerals incremented by 100. However, the second embodiment differs with respect to the shape and size of the first capsule part 14 and second capsule part 16.

The IUD 110 of the second embodiment includes a first capsule 114 that is a spherical cap that is about three-fourths a height of the spherical body 112 and a second capsule 116 that is a spherical cap that is about one-fourth the height of the spherical body 112 to allow for a more complete filling of the interior spherical cavity 118 of the spherical body 112 with fluid 50. In this respect, more weight can be added to the spherical body 112 through the ability to hold additional fluid 50.

The first capsule 114 may provide a spherical cap that is three-fourths of a sphere with a top one-fourth spherical cap of the second capsule 116 removed at a string end 140 of the spherical body 112. The interior of the first capsule 114 is hollowed out to provide an interior bowl 120.

In one embodiment, the wall 113 of the first capsule 114 may have an outer diameter between 30-35 mm and approximately 35 mm and an inner diameter between 14-31 mm and approximately 24 mm such that the thickness of the wall 113 is between 2-8 mm and approximately 5.5 mm.

The second capsule 116 may provide a spherical cap with a bottom three-fourth spherical cap removed from an insertion end opposite the string end 140. The interior of the second capsule 116 is hollowed out to provide an interior bowl 130.

In one embodiment, the wall 113 of the second capsule 116 may have an outer radius of between 15-20 mm and approximately 17.50 mm and an inner radius of between 10-15 mm and approximately 12.00 mm such that the thickness of the wall 113 is between 3-8 mm and approximately 5.5 mm.

Referring specifically to FIG. 6, in use, the larger volume interior bowl 120 of the first capsule 114 permits a greater amount of fluid 50 to be filled inside the first capsule 114 prior to the attachment of the second capsule part 16 to form the spherical body 112. Once the first capsule 114 and second capsule 116 are sealable attached, the spherical body 112 may be rotated or flipped such that the fluid flows to fill the interior spherical cavity 118 of the spherical body 112.

The spherical cavity 118 may have a volume between 0.45 ml and 1.3 ml and at least 0.45 ml and at least 0.55 ml and at least 0.65 ml and at least 0.75 ml and at least 0.85 ml and at least 0.95 ml and at least 1.05 ml and at least 1.15 ml. A fluid volume of between 0.34 ml and 0.975 ml and at least 0.34 ml and at least 0.4 ml and at least 0.5 ml and at least 0.6 ml and at least 0.7 ml and at least 0.8 ml and at least 0.9 ml may be filled into the spherical cavity 118 therefore at least one-half and at least two-thirds and at least three-fourths of the cavity volume of the spherical cavity 118 may be filled with fluid 50. It is understood that the fluid 50 of the spherical body 112 provides the desired weight to the spherical body 112, and therefore, more weight may be added to the spherical body 112 of the second embodiment by way of more fluid 50.

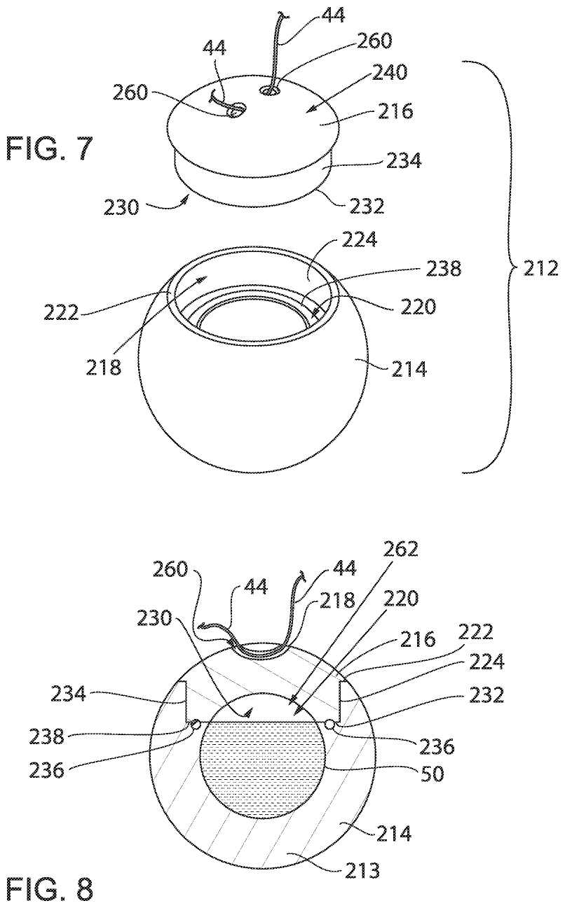

Referring to FIGS. 7 and 8, an IUD 210 of a third embodiment of the present invention is similar to the embodiments shown in FIGS. 4 through 6, where similar parts are identified by like reference numerals incremented by 100 (or incremented by 200 with respect to FIG. 1 through FIG. 3). However, the third embodiment differs with respect to the connection method of the first capsule part 114 and second capsule part 116 and the connection method of the string 44.

The second capsule part 216 may be coupled to the first capsule part 14 by a friction fit such that the lower edge 232 of the second capsule part 116 is brought into the interior bowl 220 of the first capsule part 214 and abuts an inwardly extending rim 238 of the first capsule part 214. The exterior surface 234 of the second capsule part 216 and the interior surface 224 of the upper edge 222 of the first capsule part 214 are two tight fitting mating surfaces which are pressed together to seal the first capsule part 214 and second capsule part 216 by force.

The inwardly extending rim 238 may support thereon a gasket material 236 such as an O-ring sealing the interface between the inwardly extending rim 238 of the first capsule part 214 and the lower edge 232 of the second capsule part 216. In one embodiment, the O-ring may have an outer diameter that generally corresponds to an inner diameter of the first capsule part 214 and may be between 25-20 mm and approximately 28 mm and approximately 28.3 mm. The O-ring may be made from PTFE, Nitrile (Buna), Neoprene, EPDM Rubber and Fluorocarbon (Viton) and the like.

A string end 240 of the second capsule part 216 may include a pair of openings 260 that are joined by a U-shaped channel 262 that extends into the wall 113 of the second capsule 116 a depth that is less than the thickness of the wall 113. In one embodiment of the present invention, the U-shaped channel 262 extends a depth that is less than one-half and less than one-third and less than one-fourth the thickness of the wall 113 to allow the string 44 to be threaded into one of the pair of openings 260, through the U-shaped channel 262, and out through the other of the pair of openings 260 to tie and secure the string 44 to the second capsule part 216.

The string 44 may be made from a rayon or a rayon-cotton blend material or the like. The string 44 may extend a length approximating a distance between the vagina and uterus of the mare and may be at least 10 cm and at least 20 cm in length. The string 44 may be pulled outward from the vagina to easily remove the spherical body 12 from the uterus of the mare.

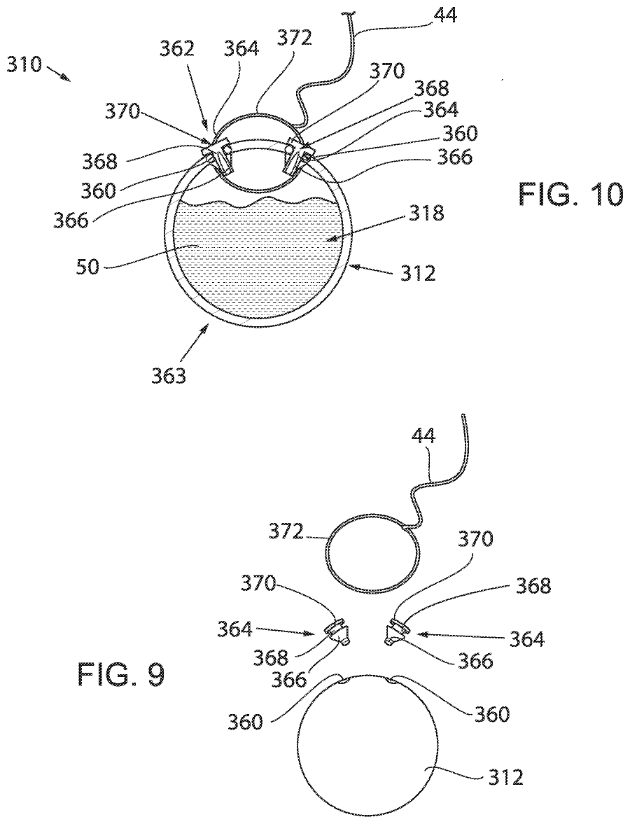

Referring to FIGS. 9 and 10, an IUD 310 of a fourth embodiment of the present invention is similar to the embodiment shown in FIGS. 1 through 3, where similar parts are identified by like reference numerals incremented by 300. However, the third embodiment differs with respect to the form of a spherical body 312 which in this embodiment is a hollowed unitary sphere instead of separate capsule parts forming the spherical body 312. The spherical body 312 may be an elastic material to assist with insertion of the spherical body 312 into the uterus.

In use, the spherical body 312 of the fourth embodiment allows for filling of a majority of the interior spherical cavity 318 of the spherical body 312 with fluid 50 through a pair of openings 360 at the string end 362 of the spherical body 312 when the string end 362 of the spherical body 312 is oriented upwards and an insertion end 363 of the spherical body 312 is oriented downwards. The interior spherical cavity 318 may be filled up to a level of the openings 360. Once the interior spherical cavity 318 has been filled with fluid 50, the openings 360 at the string end 362 of the spherical body 312 may be plugged with a pair of push plugs 364.

Each of the pair of push plugs 364 may include a tapered stopper 366 that is conical or cylindrical in shape and corresponds to a shape of the openings 360. The push plugs 364 may be made of an elastic material such as rubber, silicone, and the like that prevent the fluid 50 from leaking out from the pair of openings 360 when inserted into the openings 360.

An upper end of the push plugs 364 may support a center opening leading to a channel extending through the push plugs 364 to support the string 44 therethrough. Each of the push plugs 364 includes tapered stoppers 366 extending into the openings 360 of the spherical body 312 and supporting an upwardly extending drum 368 and an outer flange 370. The outer flange 370 generally has a larger diameter than the drum 368 and may support the center opening receiving the string 44.

In one embodiment of the present invention, with the spherical body 312 empty, a curved needle may be used to pass the string 44 through a first push plug 364 by threading the string 44 through the center opening of the outer flange 370 and out through the tapered stopper 366. Then, the curved needle may be passed through a first opening 360 of the spherical body 312 to allow the string 44 to further extend through the interior spherical cavity 318 and out through the second opening 360. Next, the curved needle is passed through the second push plug 364 through the tapered stopper 366 to extend outward through a center opening of the outer flange 370 of the second push plug 364. The push plugs 364 can then be pushed into the respective openings 360 with the string 44 extending out from the spherical body 312. The string 44 can be looped (i.e., loop 372) and tightened as necessary.

A syringe may be used to deliver fluid 50 into the interior spherical cavity 318 through the center opening of one of the installed push plugs 364 within the same channel as the string 44 extends through the plug 364. An adhesive seal may be applied on the installed push plugs 364 around the outer flange 370 to prevent leakage of the fluid 50.

Once the interior spherical cavity 318 is filled with fluid 50, the spherical body 312 may then be rotated such that the string end 362 of the spherical body 312 is oriented downwards and the insertion end 363 of the spherical body 312 is oriented upward so that the fluid 50 flows toward the string end 362 of the spherical body 312. At least one-half and at least two-thirds and at least three-fourths of the cavity volume of the spherical cavity 18 may be filled with fluid 50.

The spherical body 312 may then be inserted into the uterus of the mare with the insertion end 363 of the spherical body 312 inserted first and the string end 362 positioned proximate the uterus opening or cervix.

The string 44 may allow for the spherical body 312 to be easily removed after insertion of the spherical body 312 inside the uterus of the mare as previously described above.

It is understood that the fluid 50 assists with providing the desired weight to the spherical body 312. The fluid 50 may be a heavy, high density fluid providing additional weight to the spherical body 312.

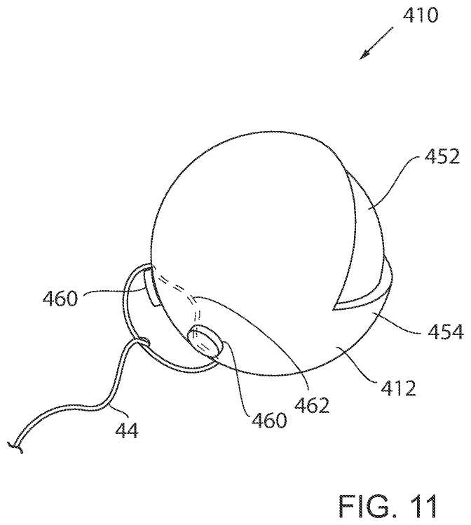

Referring to FIG. 11, an IUD 410 of a fifth embodiment of the present invention is a spherical body 412 that is composed of an interior weighted ball 452 coated or covered by an outer medical grade plastic 454 on an exterior of the ball.

The interior weighted ball 452 may be made out of a heavy, hard material, such as wood and the like. The outer medical grade plastic 454 may be made out of a biocompatible and medical grade plastic material such as PVC, Polyethylene, PEEK, Polycarbonate, Ultem PEI, Polysulfone, Polypropylene and Polyurethane and the like.

The spherical body 412 may be similarly sized as the embodiments shown and described in FIGS. 1 through 8. In this respect, the spherical body 412 may have an outer diameter of between 25-40 mm and approximately 35 mm. It is understood that the spherical body 412 may alternatively take an oblong or oval shape. It is understood that the interior weighted ball 452 provides the desired weight to the spherical body 412 instead of fluid 50.

Similar to the embodiments shown and described in FIGS. 1 through 10, a string 44 may be attached to the spherical body 412 to allow the spherical body 412 to be easily removed after insertion of the spherical body 412 inside the uterus of the mare as previously described above.

The string 44 may be attached to the outer medical grade plastic 454 using a pair of openings 460 that are joined by a U-shaped channel 464 that extends into the outer medical grade plastic 454 a depth that is less than the thickness of the outer medical grade plastic 454. In this respect, the string 44 can be threaded through into one of the pair of openings 460, through the U-shaped channel 464, and out through the other of the pair of openings 460 to tie and secure the string 44 to the outer medical grade plastic 454.

In an alternative embodiment of the present invention, the string 44 may be attached to the outer medical grade plastic 454 using an adhesive such as epoxy, acrylate, silicones and the like.

Certain terminology is used herein for purposes of reference only, and thus is not intended to be limiting. For example, terms such as “upper,” “lower,” “above,” and “below,” refer to directions in the drawings to which reference is made. Terms such as “front,” “back,” “rear,” “bottom,” and “side,” describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first,” “second,” and other such numerical terms referring to structures do not imply a sequence or order unless clearly indicated by the context.

When introducing elements or features of the present disclosure and the exemplary embodiments, the articles “a,” “an,” “the,” and “said,” are intended to mean that there are one or more of such elements or features. The terms “comprising”, “including,” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

What we claim is:

1. An intrauterine device for insertion into a uterus of a mare to regulate estrous cycle comprising:
a hollow ellipsoid having a cavity and sized to be insertable into the uterus of the mare;
a string attached to the ellipsoid; and
a filler filled within the cavity of the hollow ellipsoid wherein the filler is denser than a material of the hollow ellipsoid;
wherein the ellipsoid has a greatest outer diameter between 25 mm and 40 mm and a weight of at least 4 g.

2. The intrauterine device of claim 1 wherein the cavity has a cavity volume of at least 0.45 ml.

3. The intrauterine device of claim 1 wherein the filler is a fluid having a liquid volume of at least 0.2 ml.

4. The intrauterine device of claim 1 wherein the filler has a density of at least 1 g/ml.

5. The intrauterine device of claim 1 wherein the filler is selected from the group consisting of medical grade silicone, wood, water, saline solution, and brine.

6. The intrauterine device of claim 1 wherein the string has a length of at least 10 cm.

7. The intrauterine device of claim 1 wherein the string is selected from the group consisting of rayon and rayon-cotton blend.

8. The intrauterine device of claim 1 wherein the hollow ellipsoid comprises at least two joinable components.

9. The intrauterine device of claim 8 wherein the hollow ellipsoid is a sphere and the at least two joinable components are spherical caps.

10. The intrauterine device of claim 9 wherein the spherical caps are approximately equal halves of a sphere.

11. The intrauterine device of claim 8 further comprising a rubber gasket positioned between a space between mating surfaces of the two joinable components.

12. The intrauterine device of claim 8 wherein the at least two joinable components are joinable by a first set of screw threads of a first of the at least two joinable components and a second set of screw threads of a second of the at least two joinable components wherein the first and second set of screw threads are mating threads.

13. The intrauterine device of claim 8 wherein the two joinable components are joinable through a friction fit between mating surfaces of the two joinable components.

14. The intrauterine device of claim 1 wherein the hollow ellipsoid is a unitary body.

15. The intrauterine device of claim 1 wherein the hollow ellipsoid has at least one hole extending through a wall of the hollow ellipsoid.

16. The intrauterine device of claim 1 wherein the at least one hole extends to the cavity of the hollow ellipsoid and is closeable by at least one plug.

17. The intrauterine device of claim 1 further comprising a loop attached to an outer surface of the ellipsoid

18. The intrauterine device of claim 1 wherein a wall of the hollow ellipsoid has a thickness that is between 1.5 mm and 8 mm.

19. The intrauterine device of claim 1 wherein the ellipsoid is selected from the group consisting of PVC, Polyethylene, PEEK, Polycarbonate, Ultem PEI, Polysulfone, Polypropylene and Polyurethane.

20. A method of inserting an intrauterine device into a uterus of a mare comprising:
providing a hollow ellipsoid insertable into the uterus of the mare; a string fixedly attached to the ellipsoid and facilitated removal of the ellipsoid from the uterus of the mare; a fluid filled within a cavity of the hollow ellipsoid and having a weight wherein the ellipsoid has a greatest outer diameter between 25 and 40 mm;
filling the fluid within the cavity of the hollow ellipsoid;
inserting the hollow ellipsoid into the uterus of the mare; and
pulling on the string of the hollow ellipsoid to remove the hollow ellipsoid from the uterus of the mare.
